(19) United States
(12) Patent Application Publication
(10) Pub. No.: US 2007/0287727 A1
(43) Pub. Date: Dec. 13, 2007

(54) Anti-Nicotine Treatment

(76) Inventor: Jacob Hiller, Morges (CH)

Correspondence Address: JOHN S. PRATT, ESQ;KILPATRICK STOCKTON, LLP, 1100 PEACHTREE STREET, ATLANTA, GA, 30309, US

(21) Appl. No.: 11/757,830

(22) Filed: Jun. 4, 2007

Related U.S. Application Data
(60) Provisional application No. 60/804,211, filed on Jun. 8, 2006.

(51) Int. Cl. A61K 31/46

(52) U.S. Cl. 514/304

(57) ABSTRACT

A method is described for treatment of nicotine dependency and cessation or reduction of tobacco use in humans which combines pharmacotherapy designed to ameliorate the initial withdrawal symptoms followed by behavioral modification intended to minimize the relapse rate. Pharmacotherapy consists of the use of anticholinergic agents, which facilitate a smooth withdrawal from nicotine. A composition is described comprising scopolamine, glycopyrrolate and benztropine which is injected parenterally. In addition the individuals participate in weekly counseling sessions for at least 4 weeks after treatment.

### PRIOR RELATED APPLICATIONS

This application claims the priority benefit of U.S. Provisional Patent Application No. 60/804,211 filed Jun. 8, 2006.

### FIELD OF THE INVENTION

This invention relates to the use of pharmaceutical compositions that contain anticholinergic agents as an anti-nicotine treatment for use in treating nicotine dependency.

### BACKGROUND

Diseases related to tobacco use, such as lung disease, heart disease and cancer, claim an estimated 400,000 lives each year. The combustion or chewing of tobacco products produces poisons and carcinogens that present a significant health hazard for tobacco users. Nicotine is a principal component of tobacco, and the most pharmacologically active component. It is physically addictive, making it extremely difficult for a smoker to quit.

Smoking a cigarette delivers nicotine-containing vapors to the lungs, where the nicotine is rapidly absorbed into the bloodstream and delivered to the brain. Nicotine interacts with nicotinic acetylcholine receptors in the brain to induce the release of neurotransmitters and produce an immediate reward—the “rush” that smokers experience. A persistent stimulus is also produced that is associated with a high blood level of nicotine. As such, the dopaminergic reward system is activated which eventually results in nicotine dependency. Complex behavioral and social aspects of smoking, e.g., the hand-to-mouth ritual, etc., are also habit-forming.

Smokeless or “chewing” tobacco releases nicotine into the saliva which is absorbed through the mucous membranes in the mouth along with numerous toxins and carcinogens. An examination of the pharmacokinetics of nicotine (i.e., nicotine absorption, distribution, and elimination) resulting from smoking and smokeless tobacco use indicates that the magnitude of nicotine exposure is similar for both. Smokeless tobacco contains over 2,000 chemicals, many of which have been directly related to causing cancer. According to the Centers for Disease Control and Prevention, each year, about 30,000 Americans learn they have mouth and throat cancers, and nearly 8,000 Americans die of these diseases. Since the exposure to nicotine from smokeless tobacco is similar in magnitude to nicotine exposure from cigarette smoking, the health consequences of smoking that are caused by nicotine also would be expected to be hazards of smokeless tobacco use. Further diseases in which nicotine may play a contributory or supportive role include coronary artery and peripheral vascular disease, hypertension, and peptic ulcer disease. People who chew tobacco are 20 percent more likely to be killed by a heart attack or stroke than nonusers.

With chronic nicotine use, biochemical tolerance and dependency are developed at specific parasympathetic neuroreceptor sites by increased acetylcholine accumulation mediated via enzyme induction and/or de-repression through choline acetyltransferase.

Mammalian studies on the superior cervical ganglia show that chronic nicotine treatment causes acetylcholine increase of about 35%. Marked neurotransmitter changes occur after withdrawal of nicotine resulting in the reduction of acetylcholine accumulation even to sub-normal levels, normalization of choline acetyltransferase, and an increase of acetylcholinesterase to about 117% of controls. This activity results from the cessation of nicotine stimulation and an increased release of acetylcholine from nerve axons.

A “nicotine withdrawal syndrome” occurs from the elimination of the nicotine blockage at specific nicotinic-cholinergic synapses. Tolerance and dependency developed by increased acetylcholine synthesis are now replaced by withdrawal, which may be brought about by excessive acetylcholine stimulation. Cessation of tobacco smoking manifests symptoms such as a decrease in heart rate and blood pressure, increased irritability, anxiety, gastro-intestinal disturbances, changes in the electroencephalogram (EEG) and lack of concentration. Other symptoms include impatience, hostility, depressed mood, restlessness, and increased appetite or weight gain.

In response to nicotine withdrawal symptoms, the dependent individual commonly resumes his nicotine titration for immediate relief and returns to his prior state of “normality.” Glick et al. tested a variety of anti-cholinergic and other drugs and found that only scopolamine and d-amphetamine decreased smoking (puffing pattern) in monkeys. (Glick et al., Nature, Aug. 29, 1970; 227: 969-71).

A variety of methods to stop nicotine addiction have been tried including hypnotism, psychotherapy, electroshock aversion, and group counseling. A recent assessment of the success of smoking programs and clinics shows that fewer than half of the smokers participating in such programs quit and less than 25%-30% remain non-smokers 9-18 months later. (Evan et al. Atherosclerosis Reviews, 1979; 6:201-241).

In the past 10 years much progress has been made in the understanding of the biochemical events occurring in the brain in addiction to nicotine (Nunn-Thompson and Simon, 1989, Clinical Pharmacy, 8:710-720). Understanding the process in which several neurotransmitters are involved that simulate the rewarding effects experienced by smokers has led to emergence of pharmacological agents aimed at relieving the nicotine withdrawal symptoms (Pomerleau and Pomerleau, 1984, Neurosci. Biobehav. Rev., 8:503; Volkow et al., Nature, 386:827-830). These agents act in one of three ways: 1) Agonistic agents that include nicotine substitutes that deliver nicotine by means of chewing gum, skin patch, nasal spray and inhaler (Pomerleau and Pomerleau, 1988, Introduction; In Nicotine Replacement: A Critical Evaulation, Eds. Pomerleau and Pomerleau, New York: Liss; Palmer et al., 1922; Drugs 44:498-529; Perkins et al., 1992, Clin. Phamacol. Ther., 52:627-34; Leischow 1994, Health Value, 18:4-9). Nicotine is absorbed via the oral mucosa, skin or respiratory system to diminish the craving for nicotine enabling gradual reduction of smoking; 2) antagonistic agents that block the effects of nicotine on the cholinergic system in the periphery and the brain. These agents include, for example, mecamylamine, scopolamine and atropine (Tennant et al., 1984, NIDA Res. Monogr. 49:239-246; Rose et al., 1994, Clin. Pharmacol. Ther., 56:86-99; U.S. Pat. No. 4,555,397). Mecamylamine combined with nicotine skin patch facilitated smoking cessation beyond nicotine patch treatment alone (Rose et al, 1994, Clin. Pharmacol. Ther., 56:86-99); and, 3) agents that deliver symptomatic relief of the withdrawal symptoms from nicotine. These include clonidine (Bachynsky, 1986, Int. J. Addictions, 21(7):789-805), tricyclic antidepressants (Edwards et al., 1989, Am. J. Psychiatry, 146:373-376) and Buspiron (West et al., 1991, Psychopharmacology (Berl), 104:91-96). Recently Bupropion (Ferry et al., 1992, Circulation, 86:161-167) has been added to this group.

Other studies have provided methods for overcoming smoking addiction by weaning off the nicotine exposure (e.g., U.S. Pat. Nos. 6,845,777 and 6,874,507) while others have employed cholinergic antagonists. U.S. Pat. No. 4,555,397 provides methods of administration of scopolamine and atropine to alleviate nicotine withdrawal symptoms where the effects of atropine were potentiated by chlorpromazine.

There exists a need for therapies to lessen the symptoms of nicotine withdrawal and break the cycle of nicotine dependency that causes these individuals to relapse.

### SUMMARY OF THE INVENTION

A method is described for treatment of nicotine addition and cessation of tobacco use in individuals which combines pharmacotherapy designed to reduce the initial withdrawal symptoms followed by behavioral modification intended to minimize the long-term relapse rate. Pharmacotherapy consists of the use of anticholinergic agents, which act on the muscarinic receptors in the cerebral cortex to decrease acetylcholine neurotransmitter expression, thus enabling a smooth withdrawal from nicotine.

A composition of anticholinergic agents is administered to reduce to symptoms of nicotine dependency during cessation of tobacco use. Suitable anticholinergic agents include, but are not limited to, belladonna alkaloids, semi-synthetic derivatives of belladonna, synthetic antimuscarinic drugs, cycloplegic mydriatics and anti-Parkinson's drugs. In one embodiment, scopolamine, glycopyrrolate and benztropine are administered to reduce the symptoms of nicotine dependency during cessation of tobacco use. Pretreatment with atropine and scopolamine may increase the efficacy of the composition. Drug administration may be parenteral or transdermal, including but limited to subcutaneously and intramuscularly. Benztropine may also be administered orally.

The present invention includes transdermal patches comprising at least two anticholinergic agents which may be used in the present methods. In one aspect, the at least two anticholinergic agents for inclusion in the patch are chosen from scopolamine, glycopyrrolate and benztropine. In one embodiment, the anticholinergic agents for inclusion in the patch are scopolamine, glycopyrrolate and benztropine.

The treatment protocol is accompanied by support counseling for 4 weeks post treatment to ensure long term cessation of the tobacco product during the critical time period immediately after the treatment when withdrawal symptoms are greatest. Administration of the composition of the present invention and the ensuing treatment protocol alleviates withdrawal symptoms attributed to the cessation of chronic nicotine blockage.

It is therefore an object of the present invention to provide a composition for administration to an individual with nicotine dependency to alleviate the symptoms of nicotine withdrawal.

It is a further object of the present invention to provide a method of treatment for an individual with nicotine dependency to alleviate or ameliorate symptoms of nicotine withdrawal.

It is a further object of the present invention to provide improved treatment methods for nicotine dependency that overcome short term cravings that result in a patient resuming use of tobacco products.

It is another object of the present invention to provide a method to facilitate cessation of tobacco use in individuals who use tobacco.

It is another object of the present invention to provide a method to facilitate cessation of smoking in individuals who smoke.

Another object of the present invention is to provide a composition for administration to individuals who smoke to facilitate cessation of smoking in these individuals.

Yet another object of the present invention is to provide a method for treating symptoms of nicotine dependency in smokers by administering a combination of anti-cholinergic agents.

These and other objects, features and advantages of the present invention will become apparent after a review of the following detailed description of the disclosed embodiments and claims.

### BRIEF DESCRIPTION OF DRAWINGS

FIG. 1 provides a bar graph depicting the reasons for choosing to quit smoking by the participants in the study of the method of the present invention.

FIG. 2 provides a bar graph depicting a summary of the results of baseline spirometry measurements in the participants of the smoking cessation study.

FIG. 3 provides a bar graph depicting the dependency scores of the participants of the smoking cessation study. A score of 7 or higher indicates a high level of dependence on nicotine.

FIG. 4 provides a line graph depicting the follow up of 724 patients treated by the method of the present invention. The graph shows the percentage of treated patients remaining as non-smokers over time (months).

FIG. 5 provides a bar graph depicting the reasons for relapse in the participants in the present smoking cessation study who resumed smoking after being treated by the method of the present invention.

### DETAILED DESCRIPTION OF THE INVENTION

Definitions

It must be noted that as used in this specification and the appended claims, the singular forms “a,” “an”, and “the” include plural referents unless the context clearly dictates otherwise. Thus, for example, reference to “an active agent” or “a pharmacologically active agent” includes a single active agent as well as two or more different active agents in combination, reference to “a carrier” includes mixtures of two or more carriers as well as a single carrier, and the like.

“Carriers” or “vehicles” as used herein refer to conventional pharmaceutically acceptable excipient materials suitable for drug administration, and include any such materials known in the art that are nontoxic and fail to interact with other components of a pharmaceutical composition or drug delivery system in a deleterious manner. Such carriers are commonly known to one of ordinary skill in the art.

The term “pharmaceutically acceptable salts” in this respect, refers to the relatively non-toxic, inorganic and organic salts of the compounds described herein. These salts can be prepared in situ during the final isolation and purification of the compound. Representative salts include the bromide, chloride, hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, naphthylate, mesylate, glucoheptonate, lactobionate, and laurylsulfonate salts and the like. (See, e.g., Berge et al., “Pharmaceutical Salts”, J. Pharm. Sci., 66:1-19 (1977).) Such salts are commonly known to one of ordinary skill in the art.

By an “effective” amount or a “therapeutically effective amount” of a drug or pharmacologically active agent is meant a nontoxic but sufficient amount of the drug or agent to provide the desired effect. In the combination therapy of the present invention, an “effective amount” of one component of the combination is the amount of that compound that is effective to provide the desired effect when used in combination with the other components of the combination. The amount that is “effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, “treating” a patient involves prevention of a particular disorder or adverse physiological event in a susceptible individual as well as treatment of a clinically symptomatic individual.

As used herein, a “nicotine containing substance” refers to a substance that releases nicotine in a physiologically active form. Examples of nicotine containing substances include for example, but are not limited to, tobacco-containing substances, cigarettes, chewing tobacco, cigars, pipe tobacco and the like.

As used herein, “nicotine dependency” includes nicotine abuse, nicotine withdrawal syndrome and relapse.

The present invention provides novel compositions of anticholinergic agents. In one embodiment, at least three anticholinergic agents are used to treat nicotine dependency and facilitate a reduction or cessation in tobacco use.

In one embodiment, the at least three anticholinergic agents comprise scopolamine, benztropine and glycopyrrolate.

The present invention provides novel transdermal patches comprising at least two anticholinergic agents. In one embodiment, the anticholinergic agents comprise two or more of scopolamine, benztropine and glycopyrrolate.

The present invention provides for the use of anticholinergic agents to treat nicotine dependency and facilitate a reduction or cessation in tobacco use. In one embodiment, the anticholinergic agents are used in the preparation of a medicament for administration to humans to treat nicotine dependency and facilitate a reduction or cessation in tobacco use. In one embodiment, the anticholinergic agents comprise scopolamine, benztropine and glycopyrrolate.

Methods to treat nicotine dependency and facilitate a reduction or cessation in tobacco use are also a component of the present invention, wherein the methods employ anticholinergic agents. In one embodiment, the anticholinergic agents comprise scopolamine, benztropine and glycopyrrolate.

Compositions of the Present Invention

I. Active Ingredients

A. Anticholinergic Drugs

Most anticholinergic drugs interact with muscarinic cholinergic receptors in the brain, secretory glands, heart, and smooth muscle. A few, when given at high doses, are also able to block nicotinic receptors in autonomic ganglia and skeletal muscles. Anticholinergic agents are grouped below in Table I. Also useful in the compositions of the present invention are pharmaceutically acceptable salts of these drugs and mixtures thereof.

TABLE 1
Anticholinergic Agents

| | |
|---|---|
| Belladonna Alkaloids | atropine |
| | scopolamine HBr (Hyoscine HBr) |
| | L-hyoscyamine (Anaspaz) |
| | L-alkaloids of Belladonna |
| | (Belafoline) |
| | tincture of belladonna alkaloids |
| | (Belladonna Tincture, USP) |
| Semi-synthetic Derivatives of | homatropine HBr |
| Belladonna | homatropine methylbromide |
| | methscopolamine (Pamine) |
| | hyoscyamine |
| | eucatropine |
| Synthetic Antimuscarinic Drugs | anisotropine (Valpin) |
| | anisotropine with phenobarbital |
| | clindinium (Quarzan) |
| | glycopyrrolate (Robinul) |
| | hexocyclim (Tral) |
| | isopropamide (Darbid) |
| | mepenzolate (Cantil) |
| | methantheline (Banthine) |
| | oxyphencyclimine (Daricon) |
| | propantheline (Pro-Banthine) |
| | tridihexethyl (Pathilon) |
| | dicyclomine (Bentyl) |
| Cycloplegic Mydriatics | cyclopentolate (Cyclogyl) |
| | tropicamide (Mydriacyl) |
| Anti-Parkinson's Drugs | trihexyphenidyl (Artane) |
| | benztropine (Cogentin) |
| | orphenadrine HCl (Disipal) |
| | ethopropazine (Parsidol) |
| | diphenhydramine (Benadryl) |
| | cycrimine (Pagitane) |
| | biperiden (Akineton) |

Other drugs include antihistamines, tricyclic antidepressants and antipsychotic drugs that have anticholinergic effects. There may also be included within the scope of the invention antimuscarinic agents or anticholinergic agents such as those disclosed in U.S. Pat. Nos. 4,824,676, 4,720,494, 4,668,684, and 4,788,063. Specific anticholinergic agents are used in specific embodiments of the present invention. These agents are described in further detail as follows.

i) Atropine

Atropine is a competitive muscarinic antagonist and is a racemic mixture of D-hyoscyamine and L-hyoscyamine, with most of its physiological effects due to the L isomer. The most common atropine compound used in medicine is atropine sulphate, (C₁₇H₂₃NO₃)₂—H₂SO₄.H₂O.

In one embodiment, atropine is administered between about 1 μg/kg (kg refers to body weight (bw)) and 500 μg/kg. In a further embodiment, atropine is administered between about 5 μg/kg and 50 μg/kg. In still a further embodiment, atropine is administered at about 10 μg/kg. Initial single doses in adults vary from about 0.5 mg to 4 mg. Atropine may optionally be administered prior to administration of a composition comprising anti-cholinergic agents to treat symptoms of nicotine dependency. The effective amount of compound is routinely titrated in view of factors such as the individual's age, body mass, and tobacco use habits (e.g. number of cigarettes smoked per day).

ii) Scopolamine

Scopolamine hydrobromide is a muscarinic cholinergic antagonist with inhibitory effect on M2-cholinergic receptors of the excitatory type, that is known to inhibit the cerebral cortex, causing sedation and antivertigo, inhibit the secretion of the respiratory tract and lacrimal gland, provide antispasmodic activity and analgesia, and anti-Parkinsonism activity and platycoria.

In one embodiment, scopolamine is administered at a dose of about half the dosage of atropine that is administered. In another embodiment, scopolamine is administered between about 0.01 μg/kg and 100 μg/kg. In other embodiments scopolamine is administered at a dose between about 0.03 μg/kg and 5 μg/kg. In still other embodiments, scopolamine is administered at about 1.0 μg/kg. Initial single doses of scopolamine in adults vary from about 10 μg to about 200 μg.

iii) Glycopyrrolate

Glycopyrrolate (ROBINUL®, ROBINUL®FORTE, Wyeth-Ayerst, Madison, N.J.) is a synthetic muscarinic receptor antagonist that is known to induce smooth muscle relaxation; reduce volume and free acidity of gastric secretions, and control pharyngeal, tracheal and bronchial secretions. It antagonizes muscarinic symptoms (bronchorrhea, bronchopasm, bradycardia, and intestinal hypermotility) induced by anticholinesterases. Glycopyrrolate fails to cross the blood-brain barrier. Glycopyrrolate contains a quaternary amino functional group, and thus is capable of forming pharmaceutically acceptable salts.

In one embodiment, glycopyrrolate is administered at a dosage between about 0.1 μg/kg and 50 μg/kg. In a further embodiment, glycopyrrolate is administered at a dosage between about 0.5 μg/kg and 20 μg/kg. In still a further embodiment, glycopyrrolate is administered at a dosage between about 2 μg/kg and 7 μg/kg.

iv) Benztropine

Benztropine mesylate (COGENTIN®, Merck & Co., Whitehouse Station, N.J.) is a synthetic tertiary amine compound with structural similarities to atropine and diphenhydramine. Benztropine exhibits anticholinergic, antihistaminic and local anesthetic properties. When given orally, benztropine has an onset of action of between 1 and 2 hours. When given by intramuscular or intravenous injection, the onset of action is within minutes.

Benztropine is designated chemically as 8-azabicyclo [3.2.1]octane, 3-(diphenylmethoxy)-, endo, methanesulfonate with an empirical formula of C₂₁H₂₅NO.CH₄O₃S and a molecular weight of 403.54.

Because benztropine has cumulative action, therapy should be initiated with a low dose, which may be increased gradually by 0.5 mg increments daily at 5- or 6-day intervals, to the smallest amount necessary for optimal relief without excessive adverse effects.

In one embodiment, benztropine is administered at a dosage between about 0.1 μg/kg and 30 μg/kg. In a further embodiment, benztropine is administered at a dosage between about 0.5 μg/kg and 10 μg/kg. In still a further embodiment, benztropine is administered at a dosage between about 1 μg/kg and 5 μg/kg. The dose usually fails to exceed 6 mg per day.

B. Combination of Active Ingredients

Any of the above-mentioned compounds may be administered in combination with each other. In one embodiment, three compounds are combined for administration to a patient in need of treatment. In another embodiment, the three compounds are a combination of scopolamine, glycopyrrolate and benztropine administered for treatment of symptoms of nicotine withdrawal. In still another embodiment, at least three compounds are combined for administration to a patient in need of treatment. When the combination of scopolamine, glycopyrrolate and benztropine is administered as a composition, each is in an injectable state in a pharmaceutically acceptable fluid carrier. The amount to be administered is determined and the desired amount of each drug is combined into a single injectable composition. In one embodiment, this three drug composition is administered parenterally. In a preferred embodiment, this three drug composition is administered subcutaneously or intramuscularly. In another embodiment scopolamine and glycopyrrolate are administered parenterally, preferably subcutaneously or intramuscularly, and benztropine is administered orally.

In one embodiment the combination comprises scopolamine between about 0.01 μg/kg and about 100 μg/kg, benztropine between about 0.1 μg/kg and about 30 μg/kg and glycopyrrolate between about 0.1 μg/kg and about 50 μg/kg. In another embodiment the combination comprises scopolamine between about 0.03 μg/kg and about 5 μg/kg, benztropine between about 0.5 μg/kg and about 10 μg/kg and glycopyrrolate between about 0.5 μg/kg and about 20 μg/kg. In yet a further embodiment the combination comprises scopolamine between about 0.6 μg/kg and about 1.2 μg/kg, benztropine between about 1.0 μg/kg and about 5.0 μg/kg and glycopyrrolate between about 2.0 μg/kg and about 7.0 μg/kg.

It is to be understood that any combination of these drugs at the above dosage ranges may be useful in the present method as shown by the compositions in the table below.

TABLE 2
Compositions Comprising Scopolamine, Benztropine and Glycopyrrolate

| Com- | | | |
|---|---|---|---|
| posi- | | | Glycopyrrolate |
| tion | Scopolamine dosage | Benztropine dosage | dosage |
| 1 | 0.01 μg/kg-100 μg/kg | 0.1 μg/kg-30 μg/kg | 0.1 μg/kg-50 μg/kg |
| 2 | 0.01 μg/kg-100 μg/kg | 0.1 μg/kg-30 μg/kg | 0.5 μg/kg-20 μg/kg |
| 3 | 0.01 μg/kg-100 μg/kg | 0.1 μg/kg-30 μg/kg | 2.0 μg/kg-7.0 μg/kg |
| 4 | 0.01 μg/kg-100 μg/kg | 0.5 μg/kg-10 μg/kg | 0.1 μg/kg-50 μg/kg |
| 5 | 0.01 μg/kg-100 μg/kg | 0.5 μg/kg-10 μg/kg | 0.5 μg/kg-20 μg/kg |
| 6 | 0.01 μg/kg-100 μg/kg | 0.5 μg/kg-10 μg/kg | 2.0 μg/kg-7.0 μg/kg |
| 7 | 0.01 μg/kg-100 μg/kg | 1.0 μg/kg-5.0 μg/kg | 0.1 μg/kg-50 μg/kg |
| 8 | 0.01 μg/kg-100 μg/kg | 1.0 μg/kg-5.0 μg/kg | 0.5 μg/kg-20 μg/kg |
| 9 | 0.01 μg/kg-100 μg/kg | 1.0 μg/kg-5.0 μg/kg | 2.0 μg/kg-7.0 μg/kg |
| 10 | 0.03 μg/kg-5 μg/kg | 0.1 μg/kg-30 μg/kg | 0.1 μg/kg-50 μg/kg |
| 11 | 0.03 μg/kg-5 μg/kg | 0.1 μg/kg-30 μg/kg | 0.5 μg/kg-20 μg/kg |
| 12 | 0.03 μg/kg-5 μg/kg | 0.1 μg/kg-30 μg/kg | 2.0 μg/kg-7.0 μg/kg |
| 13 | 0.03 μg/kg-5 μg/kg | 0.5 μg/kg-10 μg/kg | 0.1 μg/kg-50 μg/kg |
| 14 | 0.03 μg/kg-5 μg/kg | 0.5 μg/kg-10 μg/kg | 0.5 μg/kg-20 μg/kg |
| 15 | 0.03 μg/kg-5 μg/kg | 0.5 μg/kg-10 μg/kg | 2.0 μg/kg-7.0 μg/kg |
| 16 | 0.03 μg/kg-5 μg/kg | 1.0 μg/kg-5.0 μg/kg | 0.1 μg/kg-50 μg/kg |
| 17 | 0.03 μg/kg-5 μg/kg | 1.0 μg/kg-5.0 μg/kg | 0.5 μg/kg-20 μg/kg |
| 18 | 0.03 μg/kg-5 μg/kg | 1.0 μg/kg-5.0 μg/kg | 2.0 μg/kg-7.0 μg/kg |
| 19 | 0.6 μg/kg-1.2 μg/kg | 0.1 μg/kg-30 μg/kg | 0.1 μg/kg-50 μg/kg |
| 20 | 0.6 μg/kg-1.2 μg/kg | 0.1 μg/kg-30 μg/kg | 0.5 μg/kg-20 μg/kg |
| 21 | 0.6 μg/kg-1.2 μg/kg | 0.1 μg/kg-30 μg/kg | 2.0 μg/kg-7.0 μg/kg |
| 22 | 0.6 μg/kg-1.2 μg/kg | 0.5 μg/kg-10 μg/kg | 0.1 μg/kg-50 μg/kg |
| 23 | 0.6 μg/kg-1.2 μg/kg | 0.5 μg/kg-10 μg/kg | 0.5 μg/kg-20 μg/kg |
| 24 | 0.6 μg/kg-1.2 μg/kg | 0.5 μg/kg-10 μg/kg | 2.0 μg/kg-7.0 μg/kg |
| 25 | 0.6 μg/kg-1.2 μg/kg | 1.0 μg/kg-5.0 μg/kg | 0.1 μg/kg-50 μg/kg |
| 26 | 0.6 μg/kg-1.2 μg/kg | 1.0 μg/kg-5.0 μg/kg | 0.5 μg/kg-20 μg/kg |
| 27 | 0.6 μg/kg-1.2 μg/kg | 1.0 μg/kg-5.0 μg/kg | 2.0 μg/kg-7.0 μg/kg |

The doses of these compounds may be routinely adjusted depending on factors such as age, body mass and smoking habits at the time of treatment. The combination of compounds may be administered separately or as a single composition in one dose. In one embodiment, the combination of compounds may be formulated into a single pharmaceutical dosage form for parenteral or oral administration. Alternatively, the combination may be formulated into a single liquid dosage form for administration in a single injection. The anti-cholinergic compounds may be administered as separate injections or separate oral dosage forms such as a tablet or caplet.

II. Pharmaceutical Compositions and Dosage Forms

Suitable routes of administration include, but are not limited to, inhalation, transdermal, oral, rectal, transmucosal, intestinal and parenteral administration, including intramuscular, subcutaneous and intravenous injections.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration may include aqueous solutions of the active compounds in water-soluble form. Additionally, suspensions of the active compounds may be prepared as appropriate oily injectable suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active compound(s) may be in powder form for reconstitution with a suitable vehicle, e.g., sterile pyrogen-free water, before use.

Transdermal drug delivery systems may also be used to administer the compositions described herein. Suitable dosage forms include creams, lotions, gels, ointments, mousses, sprays, aerosols, or any one of a variety of transdermal devices for use in the continuous administration of systematically active drugs by absorption through the skin. Additionally, transdermal patches may also be used to topically administer compositions of the present invention. Such systems dissolve or disperse the drug into a carrier composition, such as a polymeric and/or pressure-sensitive adhesive composition, from which the drug is delivered. These transdermal drug delivery systems typically are affixed adhesively to the skin or mucosa of a user, and the drug diffuses at a controlled rate from a polymer reservoir or layer into the skin or mucosa and absorbed into the blood. Such transdermal systems are described, for example, in U.S. Pat. Nos. 4,814,168, 4,994,267, 5,474,783, 5,656,286, 5,958,446, 6,024,976, and 6,905,016. The term “transdermal” is used herein in the broadest sense to refer to being able to pass through unbroken skin.

In one embodiment, a transdermal patch comprising a combination of anticholinergic compounds is used. A series of transdermal patches may be used to increase the dosage of anticholinergic compounds for administration to individuals with a history of high frequency use of cigarettes and other tobacco products. The transdermal patches in the series may be the same dosage or a series of patches of increasing dosages to gradually increase the systemic concentration of anticholingeric compounds.

In one embodiment, a transdermal patch or series of transdermal patches is provided for administering an effective amount of scopolamine, glycopyrrolate and benztropine to an individual in need of treatment. In another embodiment, a transdermal patch or series of transdermal patches in provided for administering an effective amount of a composition comprising two or more anticholinergic compounds. Additional therapeutic agents may optionally be included in the composition for transdermal delivery.

Oral dosage forms may also be used to administer the combination of active agents, and include tablets, capsules, caplets, solutions, suspensions, and/or syrups, and may also comprise a plurality of granules, beads, powders, or pellets that may or may not be encapsulated. Such dosage forms are prepared using conventional methods known to those in the field of pharmaceutical formulation and described in the pertinent texts, e.g., in Gennaro, A. R. (ed.), Remington: The Science and Practice of Pharmacy, 20th Edition (Lippincott, Williams and Wilkins, 2000). Tablets and capsules represent the most convenient oral dosage forms, in which cases solid pharmaceutical carriers are employed.

Tablets may be manufactured using standard tablet processing procedures and equipment. One method for forming tablets is by direct compression of a powdered, crystalline, or granular composition containing the active agent(s), alone or in combination with one or more carriers, additives, or the like. As an alternative to direct compression, tablets can be prepared using wet-granulation or dry-granulation processes. Tablets may also be molded rather than compressed, starting with a moist or otherwise tractable material; however, compression and granulation techniques are preferred.

In addition to the active agent(s), then, tablets prepared for oral administration using the method of the invention will generally contain other materials such as binders, diluents, lubricants, disintegrants, fillers, stabilizers, surfactants, coloring agents, and the like. Binders are used to impart cohesive qualities to a tablet, and thus ensure that the tablet remains intact after compression. Suitable binder materials include, but are not limited to, starch (including corn starch and pregelatinized starch), gelatin, sugars (including sucrose, glucose, dextrose and lactose), polyethylene glycol, waxes, and natural and synthetic gums, e.g., acacia sodium alginate, polyvinylpyrrolidone, cellulosic polymers (including hydroxypropyl cellulose, hydroxypropyl methylcellulose, methyl cellulose, ethyl cellulose, hydroxyethyl cellulose, and the like), and Veegum. Diluents are typically necessary to increase bulk so that a practical size tablet is ultimately provided. Suitable diluents include dicalcium phosphate, calcium sulfate, lactose, cellulose, kaolin, mannitol, sodium chloride, dry starch, and powdered sugar. Lubricants are used to facilitate tablet manufacture; examples of suitable lubricants include, for example, magnesium stearate, calcium stearate, and stearic acid. Disintegrants are used to facilitate disintegration of the tablet, and are generally starches, clays, celluloses, algins, gums, or crosslinked polymers. Fillers include, for example, materials such as silicon dioxide, titanium dioxide, alumina, talc, kaolin, powdered cellulose, and microcrystalline cellulose, as well as soluble materials such as mannitol, urea, sucrose, lactose, dextrose, sodium chloride, and sorbitol. Stabilizers are used to inhibit or retard drug decomposition reactions that include, by way of example, oxidative reactions. Surfactants may be anionic, cationic, amphoteric, or nonionic surface active agents.

The dosage form may also be a capsule, in which case the active agent-containing composition may be encapsulated in the form of a liquid or solid (including particulates such as granules, beads, powders, or pellets). Suitable capsules may be either hard or soft, and are generally made of gelatin, starch, or a cellulosic material, with gelatin capsules preferred. Two-piece hard gelatin capsules are preferably sealed, such as with gelatin bands or the like. See, for example, Remington: The Science and Practice of Pharmacy, cited supra, which describes materials and methods for preparing encapsulated pharmaceuticals. If the active agent-containing composition is present within the capsule in liquid form, a liquid carrier is necessary to dissolve the active agent(s). The carrier must be compatible with the capsule material and all components of the pharmaceutical composition, and must be suitable for ingestion.

When two or more active agents are combined in a single pharmaceutical dosage form, possible interactions among the active agents, and among the active agents and the excipients, must be considered. Such consideration is well within the purview of those skilled in the art of pharmaceutical formulation. The present composition thus encompasses pharmaceutical compositions wherein two or more of the active agents are separated from each other within the pharmaceutical dosage form, by, for example, separating potentially interacting compounds from each other within the pharmaceutical dosage form, as in separate flat layers of a tablet (e.g., a bilayer or trilayer tablet), concentric or other coat-type layers, coated beads or granules (which may be incorporated into a compressed tablet or into a capsule), and/or by using buffers (see, for example, U.S. Pat. No. 6,235,311). It will also be appreciated by those of ordinary skill in the art that such dosage forms, wherein two or more active agents are physically separated from the other active agents, can be manufactured so that different active agents will have different release profiles, e.g., if one active agent is formulated with an enteric coating, another active agent is formulated in a sustained release matrix, and the like. Alternatively, non-reactive pharmaceutically active derivatives of one or more of the potentially interacting compounds may be used.

Solid dosage forms, whether tablets, capsules, caplets, or particulates, may, if desired, be coated so as to provide for taste masking and/or delayed release. Dosage forms with delayed release coatings may be manufactured using standard coating procedures and equipment. Such procedures are known to those skilled in the art and described in the pertinent texts, e.g., in Remington, supra. Generally, after preparation of the solid dosage form, a delayed release coating composition is applied using a coating pan, an airless spray technique, fluidized bed coating equipment, or the like. Delayed release coating compositions comprise a polymeric material, e.g., cellulose butyrate phthalate, cellulose hydrogen phthalate, cellulose proprionate phthalate, polyvinyl acetate phthalate, cellulose acetate phthalate, cellulose acetate trimellitate, hydroxypropyl methylcellulose phthalate, hydroxypropyl methylcellulose acetate, dioxypropyl methylcellulose succinate, carboxymethyl ethylcellulose, hydroxypropyl methylcellulose acetate succinate, polymers and copolymers formed from acrylic acid, methacrylic acid, and/or esters thereof.

Sustained release dosage forms provide for drug release over an extended time period, and may or may not be delayed release. Generally, as will be appreciated by those of ordinary skill in the art, sustained release dosage forms are formulated by dispersing a drug within a matrix of a gradually bioerodible (hydrolyzable) material such as an insoluble plastic, a hydrophilic polymer, or a fatty compound, or by coating a solid, drug-containing dosage form with such a material. Insoluble plastic matrices may be comprised of, for example, polyvinyl chloride or polyethylene. Hydrophilic polymers useful for providing a sustained release coating or matrix cellulosic polymers include, without limitation: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylcellulose phthalate, cellulose hexahydrophthalate, cellulose acetate hexahydrophthalate, and carboxymethylcellulose sodium; acrylic acid polymers and copolymers, preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate, and trimethylammonioethyl methacrylate chloride (sold under the trade name Eudragit RS) preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; zein; and shellac, ammoniated shellac, shellac-acetyl alcohol, and shellac n-butyl stearate. Fatty compounds for use as a sustained release matrix material include, but are not limited to, waxes generally (e.g., carnauba wax) and glyceryl tristearate.

III. Methods of Administration

A. General Methodology

Prior to treatment, a full medical history is obtained from the patient with specific emphasis on different systemic diseases, chronic drug use and psychiatric history. Patients having a history of intraocular high pressure (Glaucoma) and benign prostatic hypertrophy (BPH) are excluded from the treatment. If a patient does not have any contraindication for treatment, such as glaucoma, BPH and acute psychiatric diseases, the patient is examined physically by the physician or other heath care professional including auscultation of the heart, lungs, checking blood pressure, pulse and intraocular pressure by the pulse tonometer. Patients are informed of the identities of the drugs to be used, dosages and possible side effects. The administration protocol and expectations for the post treatment time course are also presented and written consent is obtained. Patients are obliged to attend the treatment accompanied by another person because they are not permitted to operate an automobile for 12 hours post-treatment. Open line consultation with the doctor is available for questions or problems arising after the treatment.

An electrocardiogram is performed to comprehensively evaluate the electrical activity of the heart before and during the treatment. A test dose of a combination of low doses of anticholinergic agents is injected subcutaneously to assess the individual's response to anticholinergic treatment. In one embodiment, these agents are atropine and scopolamine. Five minutes after test injection, intraocular pressure is assessed using a pulse tonometer. If no significant changes in intraocular pressure are present, a multi-drug composition is administered subcutaneously. In one embodiment the multi-drug composition is a three drug composition of scopolamine, glycopyrrolate and benztropine. Alternatively, the drugs may be administered separately. A low dose of a local anesthetic drug is optionally provided with the anticholinergic composition. In one embodiment the local anesthetic is Marcain HCl. Other local anesthetics may also be used. Exact dosage amounts are determined based on the age, weight and smoking history of the subject.

The patient is first administered a “test dose” of anticholinergic compounds to determine the patient response to anticholinergic therapy. In one embodiment about 0.6 mg atropine with about 1.5 μg/kg scopolamine is administered to determine the patient response to anticholinergic drugs. One of ordinary skill in the art will appreciate that the any similar dosage of these compounds may be used to determine the patient response to anticholinergic treatment.

The patient is monitored closely for 10-30 minutes after administration of the initial anticholinergic composition. Patient vital signs including blood pressure, pulse and intraocular pressure are assessed to ensure that no adverse effects develop from administration of the anticholinergic composition.

If no adverse response is observed, a “treatment dose” comprising a combination of anticholinergic compounds is administered to the patient with dosages varying depending on the individual's age, weight and smoking history. In one embodiment, a composition of scopolamine, benztropine and glycopyrrolate is administered wherein the dosage used depends on the patient's history of nicotine use. Increases in benztropine and glycopyrrolate concentration between about 20-50% and 20-40% respectively may be made for individuals who have an extended history of nicotine use or have a high incidence of nicotine use. The term “heavy nicotine use” generally refers to individuals who frequently use nicotine products and as an example, would refer to individuals who smoke greater than about 25 cigarettes per day or use an equivalent amount of nicotine products. Very heavy smokers may smoke more than 40 cigarettes per day.

For example, in one embodiment a composition for administration to a moderate nicotine user would include scopolamine 1.5 μg/kg, benztropine 6.0 μg/kg, and glycopyrrolate 2.0 μg/kg, whereas the composition for administration to a heavy nicotine user would include scopolamine 1.5 μg/kg, benztropine 9.0 μg/kg, and glycopyrrolate 3.0 μg/kg. Again, one or more of these substances may be administered in combination as a single composition or separately.

After administration of the treatment dose, the patient is again monitored closely for 10-30 minutes after administration of the initial anticholinergic composition. Patient vital signs including blood pressure, pulse and intraocular pressure are assessed to ensure that no adverse effects develop from administration of the anticholinergic composition. The patient is then discharged from the clinic, into the care of an accompanying person.

The combination of the drugs using during the treatment has a high affinity to pre-synaptic cholinergic nerve terminals. While not wanting to be bound by the following statement, it is believed that the drugs act synergistically and inhibit or reduce the release of acetylcholine for 7-10 days after treatment. A widespread release of acetylcholine occurs during nicotine withdrawal. Down regulation of acetylcholine receptors or excessive acetylcholine in the synapse generally occurs within 48 to 72 hours after nicotine withdrawal. While not wanting to be bound by the following statement, it is believed that the doses of the present composition provide pharmacological blockade for more than twice the duration necessary to overcome withdrawal symptoms. Patients are permitted to contact to the clinic and arrange for further administration of the compound, if necessary, for several days post treatment, however more than 96% of the patients did not require additional treatment within the first 10 days after treatment. It is strongly recommended that patients do not consume alcohol for seven days after the treatment. Sedatives are optionally provided for the first seven days after treatment for morning and evening use. Sedatives include but are not limited to alprazolam or clorazepate dipotassium.

In one embodiment alprazolam is administered at a dosage between about 0.05 mg and about 0.5 mg. In another embodiment, alprazolam is administered at a dosage between about 0.1 mg and about 0.3 mg. In another embodiment, alprazolam is administered at a dosage of about 0.25 mg. In one embodiment clorazepate dipotassium is administered at a dosage between about 5 mg and about 75 mg. In another embodiment, clorazepate dipotassium is administered at a dosage of about 10 mg and about 50 mg. In another embodiment, clorazepate dipotassium is administered at a dosage of about 15 mg.

In one embodiment, sedatives with action on gamma amino butyric acid (GABA) receptors are used. Other sedatives that have actions on GABA receptors include, but are not limited to, alprazolam, chlordiazepoxide, clonazepam, clorazepate, diazepam, estazolam, flurazepam, lorazepam, midazolam, oxazepam, quazepam, temazepam, triazolam, halazepam, and prazepam.

In one embodiment, the sedatives are alprazolam in the morning and clorazepate dipotassium in the evening.

Heavy smokers may be defined as individuals who smoke more than 20 or 25 cigarettes per day at the time of intake. These individuals may optionally require an additional administration of the anticholinergic composition 2-5 days after initial administration as part of the treatment protocol. Patients who resume using tobacco products within 6 first months after the treatment may also be re-administered the anticholinergic composition.

Optionally, all patients are provided counseling with a psychologist beginning seven days after treatment and up to one month after the treatment. Counseling may be individual or as a group. In one embodiment, in-person counseling is provided however, telecommunications technology now allows for remote counseling as well Remote counseling includes counseling sessions where the counselor and patient are not in the same room. Such meetings may include but are not limited to telephone counseling, video conference counseling or internet chat counseling and similar modes of communication. Patients opting not to attend the counseling sessions may be provided written documentation on dealing with the psychological aspects and habits of the nicotine addiction.

The withdrawal symptoms of nicotine are severe mainly during the first 24-48 hours. In the first 48 hours the psychologist contacts each subject daily and verifies the progress of the withdrawal. Any subject who experiences severe craving to nicotine receives a booster injection. It is therefore important to suppress the symptoms in 2 weeks. In addition, the subject attends a weekly meeting guided by a psychologist lasting one hour for 4 weeks after the treatment. In these group support meetings each subject has the opportunity to share his experience with the other members of the group. The psychologist tries to resolve any problems related to the withdrawal symptoms and in some cases refers the subject to the physician. Follow up is carried out monthly for the next 6 months and then every 3 months until the end of the year.

This procedure ameliorates or prevents the physical symptoms of nicotine withdrawal and supports long-term abstinence from tobacco products without relapse. The present method provides an improvement relative to commonly used smoking cessation methods.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### EXAMPLE 1

### Treatment of Smokers Using Anticholinergic Therapy

A group of 724 smokers participated in this treatment program for 22 months; 408 (56.4%) male and 316 (43.6%) female, mean age 46.8 (standard deviation (s.d.) 9.8) yrs., average smoking period 28.2 years (s.d. 15.1) cigarettes/day for a total of 52 pack-years.

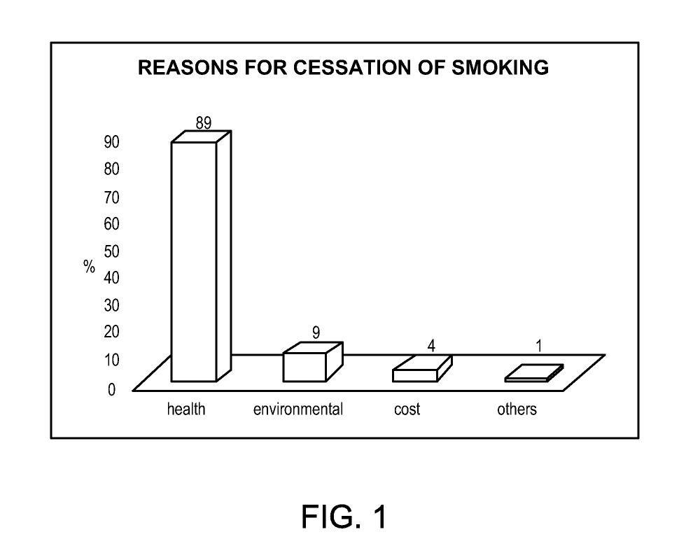

The reasons for smoking cessation varied but the greatest motivating factor was for health reasons in 86.9% of the subjects. (FIG. 1). Previous attempts to quit smoking by other means were made by 79.1% of the participants (Table 3).

TABLE 3

| | | Successful attempts for |
|---|---|---|
| Method | Successful attempts | over 3 months |
| Acupuncture | 19.7% | 4.8% |
| Hypnosis | 6.4% | 0.8% |
| Healing | 19.8% | 3.1% |
| Nicotine Substitutes | 17.9% | 5.2% |
| Psychotherapy | 6.6% | 2.3% |
| Self | 23.3% | 26.6% |

The test participants were surveyed for prior history of medical problems prior to the study. 27.1% of the participants admitted to having chronic obstructive lung disease of some sort. 13.8% of the participants were hypertensive or had coronary artery disease. 10 subjects underwent coronary artery bypass in the past (Table 4). Other diseases included diabetes in 5.4%, peptic ulcer in 4.1%, peripheral vascular disease in 3.2% and psychiatric problems in 2.3% of the participants.

TABLE 4
Self Reported Diseases (n = 709)

| Lung Diseases | % | Heart Diseases | % |
|---|---|---|---|
| Chronic Bronchitis | 4.7 | Systemic Hypertension | 9 |
| Emphysema | 1.1 | Ischemic Heart Disease | 0.4 |
| COPD | 17.5 | Others | 3 |
| Asthma | 3.8 | Post-coronary-artery bypass | 1.4 |
| Others | 4.1 | Total | 13.8 |
| Total | 31.2 | | |
| Other Diseases | | Mental Disorders | |
| Diabetes | 5.4 | Depressive States | 1.4 |
| Peptic Ulcer | 4.1 | Schizophrenia | 0.1 |
| Peripheral Vascular | 3.2 | Others | 0.8 |
| Diseases | | | |

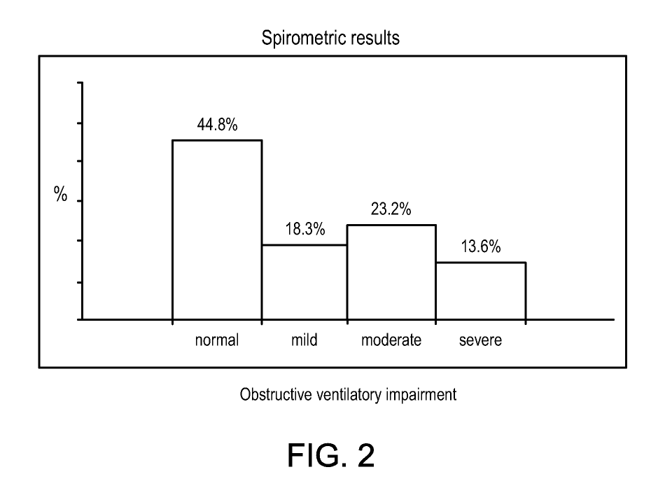

Baseline electrocardiogram and spirometry measurement were obtained prior to treatment. The electrocardiogram was abnormal in 12.3% and spirometry was abnormal in 54.4% (Table 5 and FIG. 2). Of these, 36.8% showed moderate to severe obstructive ventilatory impairment (V.I.)

TABLE 5
Laboratory Tests

| Electrocardiogram (n = 681) | % | Spirometry (n = 698) | % |
|---|---|---|---|
| Normal | 87.7 | Normal | 44.8 |
| Abnormal | 12.3 | | |
| | | Mild obstructive V.I. | 18.3 |
| | | Moderate obstructive V.I. | 23.2 |
| | | Severe obstructive V.I. | 13.6 |
| | | Total of Abnormal | 54.4 |
| | | pulmonary function | |

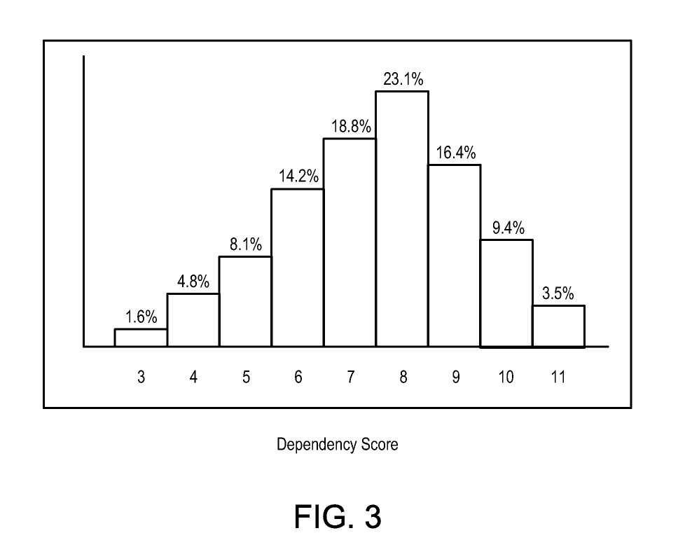

The dependency score was 7 and above in 71.2% of the subjects (FIG. 3). This score is derived from questioning the subject about their nicotine use habits and indicates a qualitative measurement of the level of nicotine dependence in a test subject. A score of 7 or higher indicates individuals with a high level of dependence on nicotine. (Fagerstrom, Addict. Behav. 1978; 3(3-4):235-41).

The treatment started with administration of 0.5 mg atropine with 0.3 to 0.8 μg scopolamine subcutaneously in saline. Intraocular pressure was measured by pulse tonometer. If no significant change in intraocular pressure occurred, scopolamine (0.6-1.2 μg/kg), glycopyrrolate (2-7 μg/kg) and benztropine (1-5 μg/kg) were all administered subcutaneously in saline along with a small dose of Marcain HCl as a local anesthetic. Exact dosage amounts were determined based on the subject's age, weight and smoking history as described previously.

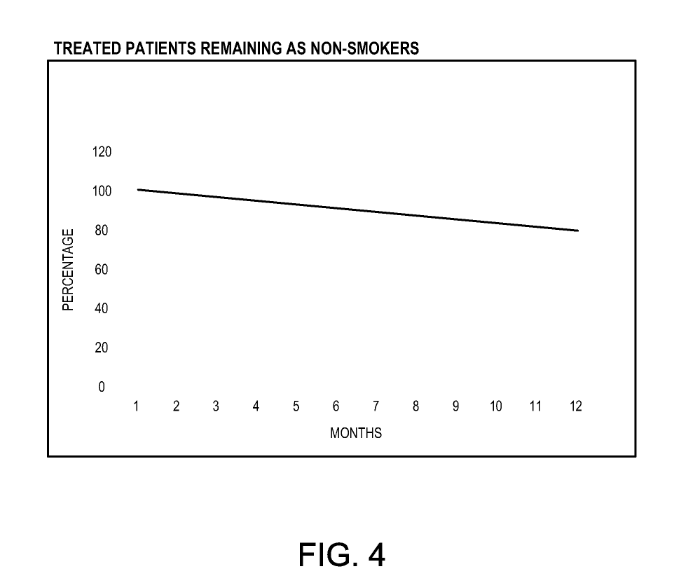

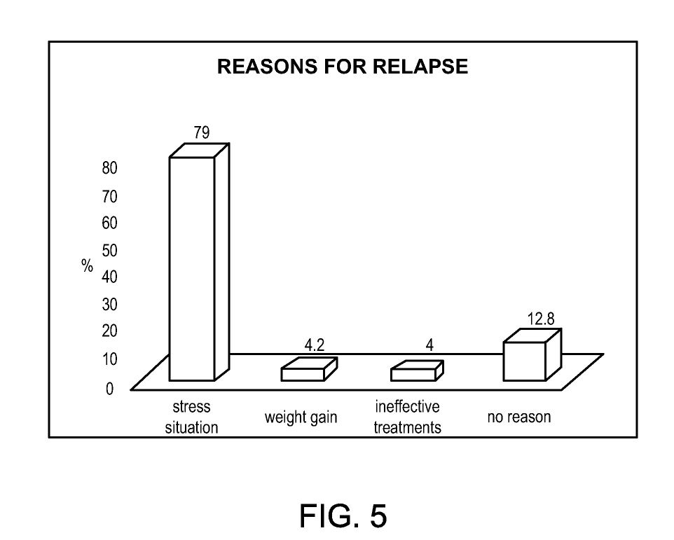

The follow up of this group was done by telephone survey. Of 724 subjects who were randomly contacted, 86.9% of this group did not smoke at the end of 2 weeks of the withdrawal, 85.4% after 2 months, 81.4% after 6 months and 77.9% were abstinent from smoking after 1 year (FIG. 4). The main reasons for resuming smoking were: recent anxiety situation, incidental resumption, ineffective treatment and insufficient motivation (FIG. 5). Some patients resumed smoking due to weight gain while others did not provide any specific reason (FIG. 5). For the majority of patients, about 87-90%, a single injection was effective. A booster injection of the treatment drug combination within the first 2 to 4 days of withdrawal was required in 10-13% of the cases, and was often used with heavy and very heavy smokers. The dosage and drug combination was the same as the original treatment dose. Side effects were mild consisting of drowsiness, sleepiness, dry mouth, blurred vision and fatigue. These symptoms subsided within 24 hours. All of these patients received normal supportive treatment by sedative drugs and counseling on coping with and adjusting to addiction.

At present it seems that cessation therapy with anticholinergic agents is the most promising method available. These data suggest that the use of anticholinergic agents in one office visit followed by behavioral support is an effective mode of therapy in smoking cessation.

### EXAMPLE 2

### Treatment of Heavy Smokers Using Anticholinergic Therapy

Some individuals were characterized as heavy smokers and smoked 20 or more cigarettes per day. These individuals have also been treated with the initial anticholinergic therapy as described in Example 1. All of these patients received normal supportive treatment by sedative drugs and counseling on coping with and adjusting to addiction.

Booster injections were offered to all patients. About 87-90% of these individuals reported that they felt good, no longer smoked, and did not need the booster injection described in Example 1. About 10-13% of the heavy smokers did need a booster injection. A percentage of the heavy smokers reported for various reasons that they were unable to visit the clinic for a booster injection. These individuals were administered tablets of Benztropin misolate (a 2 mg tablet per day), a skin patch containing 1.5 mg of Scopolamine placed on the skin of the back, or a combination of both the Benztropin tablets and scopolamine patch during the 5 days after the first injection of anticholinergic medicines. These Scopolamine patches generally provided three days of slow release of Scopolamine into the circulation. All of these patients reported feeling well a few weeks after treatment and showed no immediate or short term relapse to smoke again.

All patents, publications and abstracts cited above are incorporated herein by reference in their entirety. It should be understood that the foregoing relates only to preferred embodiments of the present invention and that numerous modifications or alterations may be made therein without departing from the spirit and the scope of the present invention as defined in the following claims.

We claim:

1. A method for treating nicotine dependence in a human in need of treatment comprising:
administering an effective amount of at least three anticholinergic agents in a pharmaceutically acceptable carrier to the human, wherein the three anticholinergic agents are administered separately or in combination.

2. The method of claim 1, wherein the three anticholinergic agents are administered in combination in one composition.

3. The method of claim 2, wherein the composition comprises scopolamine, benztropine and glycopyrrolate.

4. The method of claim 2, wherein the composition is administered parenterally or transdermally.

5. The method of claim 3, wherein the scopolamine is administered at a dosage of about 0.01 μg/kg bw to about 100 μg/kg bw.

6. The method of claim 3, wherein the scopolamine is administered at a dosage of about 0.03 μg/kg bw to about 5 μg/kg bw.

7. The method of claim 3, wherein the scopolamine is administered at a dosage of about 0.6 μg/kg bw to about 1.2 μg/kg bw.

8. The method of claim 3, wherein the benztropine is administered at a dosage of about 0.1 μg/kg bw to about 30 μg/kg bw.

9. The method of claim 3, wherein the benztropine is administered at a dosage of about 0.5 μg/kg bw to about 10.0 μg/kg bw.

10. The method of claim 3, wherein the benztropine is administered at a dosage of about 1.0 μg/kg bw to about 5.0 μg/kg bw.

11. The method of claim 3, wherein the glycopyrrolate is administered at a dosage of about 0.1 μg/kg bw to about 50.0 μg/kg bw.

12. The method of claim 3, wherein the glycopyrrolate is administered at a dosage of about 0.5 μg/kg bw to about 20.0 μg/kg bw.

13. The method of claim 3, wherein the glycopyrrolate is administered at a dosage of about 2.0 μg/kg bw to about 7.0 μg/kg bw.

14. The method of claim 1, further comprising administration of a sedative.

15. The method of claim 14, wherein the sedative is alprazolam or clorazepate dipotassium.

16. The method of claim 15, wherein the alprazolam is administered at a dosage of about 0.05 mg and to about 0.5 mg and the clorazepate dipotassium is administered at a dosage of about 5 and to about 75 mg.

17. The method of claim 15, wherein the alprazolam is administered at a dosage of about 0.1 mg to about 0.3 mg and the clorazepate dipotassium is administered at a dosage of about 10 mg to about 50 mg.

18. The method of claim 15 wherein the alprazolam is administered at a dosage of about 0.25 mg and the clorazepate dipotassium is administered at a dosage of about 15 mg.

19. The method of claim 14, wherein the sedative is administered in the morning and evening

20. The method of claim 1, further comprising post-treatment counseling.

21. The method of claim 20, wherein the post-treatment counseling is selected from in-person counseling, telephone counseling, videoconference counseling, or internet chat counseling.

22. The method of claim 1, further comprising oral administration of benztropine, transdermal administration of scopolamine, or both.

23. The method of claim 1, wherein the treatment results in a reduction or cessation of tobacco use in the human.

24. A composition comprising three anticholinergic agents in a pharmaceutically acceptable carrier.

25. The composition of claim 24 wherein the three anticholinergic agents comprise scopolamine, benztropine and glycopyrrolate.

26. A transdermal patch comprising at least two anticholinergic agents.

27. The transdermal patch of claim 26, wherein the anticholinergic agents comprise scopolamine, benztropine and glycopyrrolate.
